(19) United States
(12) Patent Application Publication
(10) Pub. No.: US 2014/0114262 A1
(43) Pub. Date: Apr. 24, 2014

(54) STICK PACK WITH APPLICATOR

(71) Applicant: Sébastien Lucien Fily, Ville Mont-Royal (CA)

(72) Inventor: Sébastien Lucien Fily, Ville Mont-Royal (CA)

(73) Assignee: BAYER CONSUMER CARE AG, Basel (CH)

(21) Appl. No.: 14/116,007

(22) PCT Filed: Apr. 30, 2012
(86) PCT No.: PCT/EP2012/057929

§ 371 Date: Jan. 10, 2014

(30) Foreign Application Priority Data
May 6, 2011 (EP) 11290216.8

(51) Int. Cl. A61M 35/00

(52) U.S. Cl. CPC A61M 35/003
USPC 604/310; 53/455

(57) ABSTRACT

Stick pack (10) for a liquid, cream or gel-type product comprising a tubular sachet (12) with a first end (14) and a second end (16); a first portion (13) close to the first end (14) being filled with liquid, cream or gel-type product; a second portion (15); an applicator insert (20) between said first end (14) and said second end (16), said applicator insert (20) being sealed to the tubular sachet (12) such that no product can pass between the wall of the tubular sachet (12) and the applicator insert (20), said applicator insert (20) having a passage (22) for the product.

The invention relates to a stick pack with an applicator insert for a single dose of a liquid, cream or gel-type product for external application to a surface such as the skin.

Stick packs as such are known from the prior art. For example EP 0 705 204 B1 discloses a stick pack for liquid medicinal products consisting of a three-seamed tubular foil sachet of aluminum foil containing an individual dose of a medicinal product. The foil sachet has a lengthwise seam and it is closed at each end by sealed transverse seams. One of the transverse seams has a lateral incision for opening.

The company Sigpack Systems AG, CH 8222 Beringen, Switzerland, discloses on its Webpage a pull pack: http://pa.bosch.com/sigpacksystems/eng/pdf/PullPack Flyer ENG.pdf

A pull pack is a stick pack consisting of two parts which are overlapping each other when the pull pack is closed. This pull pack splits into two parts upon pulling apart both ends. The pull pack is adapted to the packing of solid products.

U.S. Pat. No. 4,430,013A and U.S. Pat. No. 5,487,932A disclose packages with applicators for a single dose of fluid or foam for external application to the skin.

U.S. Pat. No. 4,430,013A discloses an applicator package having distal ends and having a foam applicator pad and a backing member adjacent thereto. The backing member has at least one reservoir and is formed of a relatively rigid, flat sheet material and a formed material for containing the contents of the reservoir there between. The flat sheet material has a slit or weakened portion in the surface of the material. The slit or weakened portion is transversely disposed of the flat sheet material, so that the slit or weakened portion is ruptured upon forcing the distal ends toward each other about the slit or weakened portion. The contents of the reservoir are thereby released into the foam applicator pad upon pressure deforming the formed material containing the reservoir. The device of U.S. Pat. No. 4,430,013A has the disadvantage that the applicator pad is not protected from dust or external contamination per se, since it is located at the outside of the device. Additional means for protection such as an envelope that has to be removed prior use are necessary.

U.S. Pat. No. 5,487,932 discloses a combined fluid storage container and applicator device for viscous fluids comprising a sheet-like impermeable material having overlying symmetrical or asymmetrical portions with a fold line and a temporary seal around the remaining periphery of the device forming a cavity for the fluid, a pad within the cavity and adhered to the sheet, and a separation mechanism for simultaneously applying continuous separating force in multiple directions to open the cavity and expose the pad. The device of U.S. Pat. No. 5,487,932 has the disadvantage that although the application pads are protected, the opening and making the applicator ready and available for usage requires sophisticated action. Further this applicator is only adapted to be used with a liquid and not with paste-like contents.

U.S. Pat. No. 5,577,851 discloses a tube dispenser for several doses of a liquid or paste-like substance with a sponge applicator and cover assembly. The tube dispenser has a main body portion, an upper neck portion extending from the main body at an upper end and an upper circumferential portion at the upper end of the main body portion adjacent to the-neck portion. A sponge applicator for attachment around the neck portion of the tube dispenser is adapted to apply any substance contained in the tube dispenser. A cover member has an upper portion for covering the sponge applicator and the neck portion of the tube dispenser and a lower circumferential portion to mate with and seal to the upper circumferential portion of the main body of the tube dispenser. The tube dispenser has three separate components, which makes it expensive to manufacture, apart from the fact that tubes are more expensive to manufacture than stick packs.

Packages including a single dose of product for application to a surface such as the skin do not usually offer an applicator or other device for applying to and/or massaging the surface to be treated. When such an applicator device is provided, it often happens that it is not protected from dust or external contamination. For some packages, such a protection for the applicator is delivered, but with a sophisticated way of opening the package and making the applicator ready and available for usage. Last but not least known devices such as the tube dispenser of U.S. Pat. No. 5,577,851 are costly to produce, because they require a lot of components which have to be assembled together after filling the tube.

The solution of the problems mentioned above is a combination of applicator and massaging device with the stick-pack technology (i.e. 3-side sealed pack, one longitudinal sealing to form a tube, and two horizontal sealings to close the tube). Stick packs are typically made from a multi layer foil. According to the invention this multi layer foil includes at least a barrier layer made of a barrier material such as aluminum or ethylene vinyl alcohol (EVOH), and a sealing layer on one side, the inner side of the stick pack, of the barrier layer. The sealing layer may include linear low-density polyethylene (L-LDPE) or low-density polyethylene (LDPE) or ionomer resin (like Surlyn®) or ethylene-acrylic acid (EAA) resin (like Nucrel®). Optionally the barrier layer may have a third layer on the other side, the outer side of the stick pack. The third layer may be made from polyethylene terephthalate (PET) or paper and have colors, letters, symbols or the like printed on it.

The stick pack for a liquid, cream or gel-type product according to the invention comprises a tubular sachet with a first end and a second end; a first portion close to the first end is filled with liquid or gel-type product; a second portion; an applicator insert between said first end and said second end, said applicator insert being sealed to the tubular sachet such that no product can pass between the inner wall of the tubular sachet and the applicator insert, said applicator insert having a passage for the product. The passage can have capillary dimensions, such that the product stored in the second portion can move through the passage by capillary action. It is also possible that the passage has non-capillary dimensions such that product is moved through the passage by compressing the first portion of the tubular sachet.

Preferably the applicator insert is made of the same or similar material as the sealing layer, especially high-density polyethylene (HDPE) or low-density polyethylene (LDPE).

In one embodiment the tubular sachet has a circumferential area where the sachet is adapted to be broken to separate the second portion of the tubular sachet from the first portion. The circumferential area is located between the applicator insert and the second end. Preferably the circumferential area is located close to the applicator insert. The circumferential area can be a weak area for example with pre-cuts in one or more of the layer of the multi layer foil forming the tubular sachet.

In another embodiment of the invention the applicator insert is equipped with a sponge (synthetic or natural), cotton pad or other porous material, or with a silicone part that will support the application of the liquid or gel-type product.

In one embodiment the applicator insert is of cylindrical shape and has a top wall including the passage for the product.

Depending on the viscosity of the liquid, cream or gel-type product to be dispensed, a valve, i.e. a silicone valve can be located adjacent or in the passage for the product at the applicator insert so that it requires a certain amount of pressure to open the insert and to release the liquid, cream or gel-type product on the applicator side. The valve is adapted to come back to its closed position once the pressure is released on the first portion of the stick pack that includes the liquid, cream or gel-type product.

When manufacturing the stick pack of the present invention a tubular foil is closed at one end by sealing thus forming a tubular sachet closed at one end. The tubular sachet is filled with a liquid or gel-type product. After that an applicator insert is sealed to the tubular sachet adjacent the liquid or gel-type product opposite the closed end of the tubular sachet. The applicator insert is sealed along its periphery to the tubular foil such that no product can pass between the tubular foil and the applicator insert. Together with sealing the insert or thereafter the remaining open end of the tubular sachet is sealed adjacent the applicator insert.

To use the stick pack according to the invention the user has to pull on both stick pack ends so that the stick pack is separated in two parts. The first part including the liquid or gel-type product, and the applicator insert having a passage from one side of the applicator insert to the other. Then the user uses the unit dose of liquid or gel-type product within the first part of the stick pack by squeezing the stick pack, such that the liquid or gel-type product is forced through the passage in the applicator insert and can be applied. The second part of the stick pack can be wasted.

It is more convenient for the user to apply a liquid, cream or gel-type product such as a cream or an ointment from a stick pack with the aid of an applicator then from a basic square 4-side sealed sachet or 3-side sealed stick pack directly. The positioning in hand of a stick-pack with its applicator is very similar to the handling of a pen; it makes the usage more convenient and accurate.

### FIGURES AND EXAMPLES

FIG. 1 shows a first cross sectional view of the stick pack with applicator

FIG. 2 shows a second cross sectional view of the stick pack with applicator

FIG. 3 shows a side view of the stick pack with applicator with the applicator insert in dotted lines

FIG. 4 shows a perspective view of the stick pack with applicator

FIG. 5a-c shows the usage of the stick pack with applicator

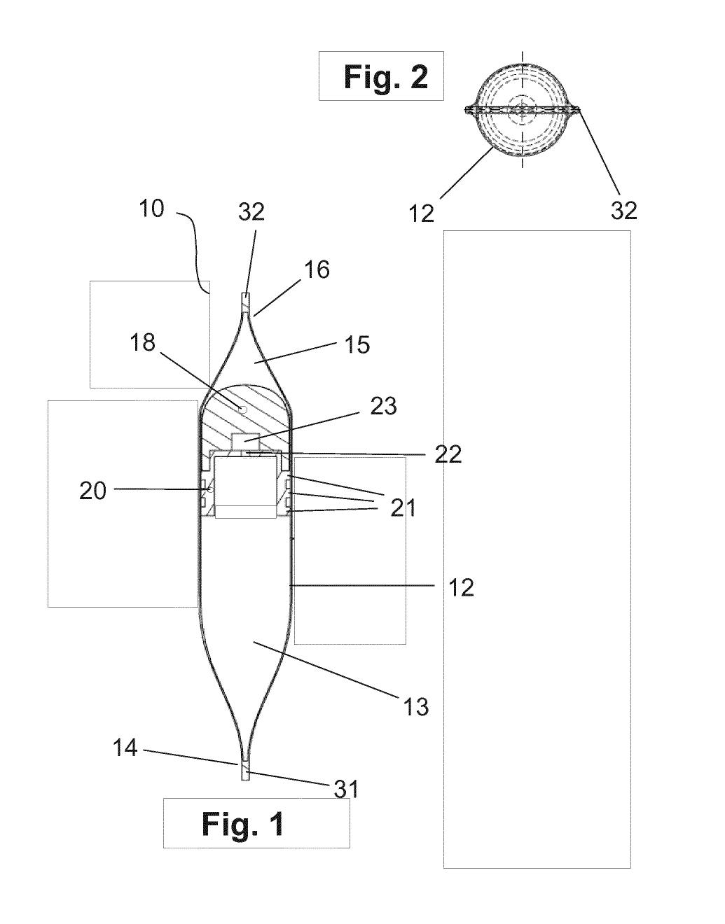

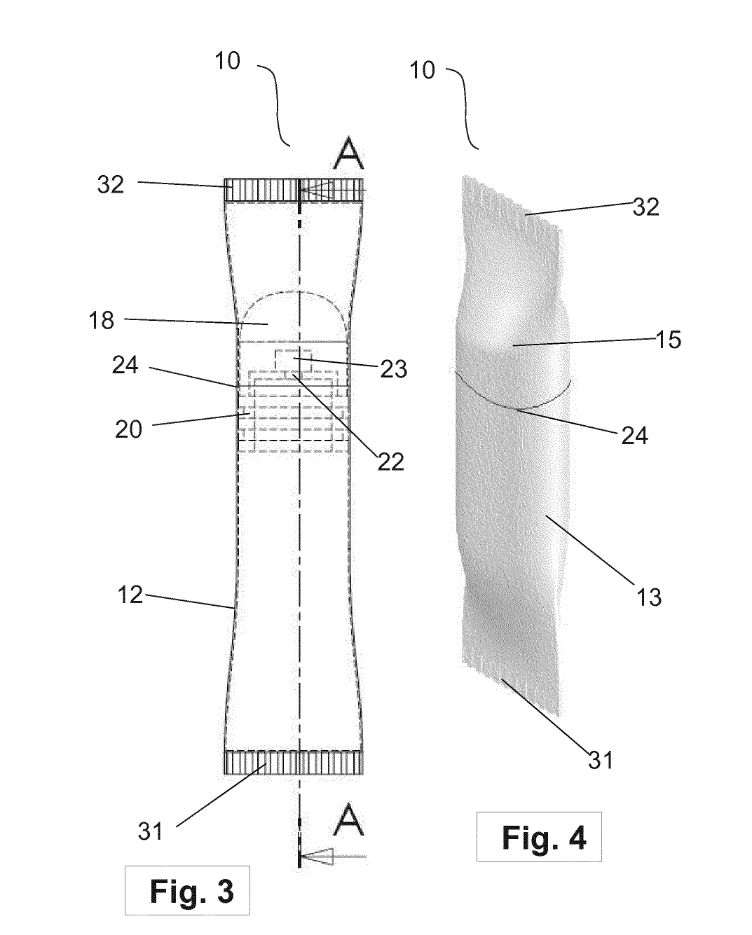

FIGS. 1 to 4 show different views of one example of the stick pack with applicator 10 according to the invention. FIG. 1 is a cross sectional view of the stick pack with applicator 10 along line A-A shown on FIG. 3. The tubular sachet 12 is formed from multi layer foil by longitudinal sealing i.e. parallel line A-A as shown in FIG. 3, to form a tube and has a sealed first end 14 and a sealed second end 16. The first seal 31 at the first end 14 is made prior to filling of the stick pack. The second seal 32 at the second end 16 is made after filling the tubular sachet 12 with a liquid, cream or gel-type product and after or at the same time with the sealing of the applicator insert 20 to the tubular sachet 12.

The applicator insert 20 has a generally cylindrical shape with circumferential sealing ribs 21 on its outer surface. The applicator insert 20 has a top wall with a passage 22 for the product. A valve 23 is attached to the top wall above the passage. The applicator insert 20 is sealed to the tubular sachet 12 such that the sealing ribs form a tight seal with the inner layer of the tubular sachet 12 and no product can pass between the inner wall of the tubular sachet 12 and the applicator insert 20. The applicator 18 made of a porous material like a sponge or a compacted wool applicator or the like, is attached to the top of the applicator insert 20 by gluing. The applicator 18 can be attached to the applicator insert by any other means known to the person skilled in the art such as welding, heat-sealing or screwing.

A circumferential pre-cut 24 is present in the outer layer of the multi layer foil of the tubular sachet 12, which divides the first portion 13 of the tubular sachet 12 from the second portion 15. The first portion 13 includes the product and the applicator insert 20 and is closed by a first seal 31 on the first end 14. The second portion 15 is closed by the second seal 32 on one end. The second portion 15 serves as a protective cover for the applicator 18 while attached to the first portion 13 and is discarded after being removed from the first portion 13.

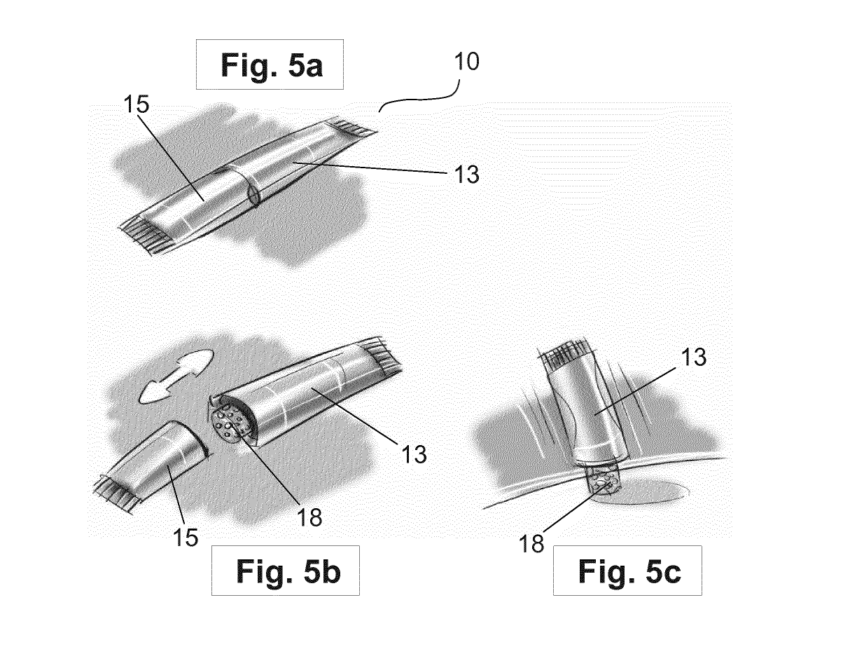

FIG. 5 a-c show how the stick pack 10 is being used. The user has to pull on both stick pack portions 13 and 15 so that the stick pack 10 is separated into the two portions 13 and 15. The first portion 13 includes the liquid, cream or gel-type product and the applicator insert 20 with the applicator 18. The user uses the unit dose of liquid or gel-type product within the first portion 13 of the stick pack 10 by squeezing the stick pack 10, such that the product is forced through the passage 22 in the applicator insert and can be applied. The second portion 15 of the stick pack 10 can be wasted.

1. Stick pack for a liquid, cream or gel-type product comprising
a tubular sachet with a first end and a second end;
a first portion close to the first end being filled with liquid, cream or gel-type product;
a second portion;
an applicator insert between said first end and said second end, said applicator insert being sealed to the tubular sachet such that no product can pass between the wall of the tubular sachet and the applicator insert, said applicator insert having a passage for the product.

2. Stick pack of claim 1, wherein the tubular sachet has a circumferential area where the tubular sachet is adapted to be broken to separate the second portion from the first portion, said circumferential area located between the applicator insert and said second end.

3. Stick pack of claim 2, wherein the circumferential area is located close to the applicator insert.

4. Stick pack of claim 1, wherein the applicator insert is of cylindrical shape and has a top wall including the passage for the product.

5. Stick pack of claim 1, wherein the applicator insert is equipped with an applicator made of a porous material such as sponge or cotton pad or made as a silicone part.

6. Stick pack of claim 1, wherein a valve is located in the passage for the product or at the top wall of the applicator insert adjacent the passage.

7. Stick pack of claim 6, wherein the valve is a silicon valve.

8. Stick pack of claim 1, wherein the tubular sachet has one longitudinal sealing to form a tube of a foil and two horizontal sealings at the first and second ends.

9. Method for manufacturing a liquid, cream or gel-type product comprising the steps of
providing a tubular foil;
closing said tubular foil at one end thus forming a tubular sachet being open at one end;
filling said tubular sachet with a liquid, cream or gel-type product;
sealing an applicator insert to the tubular sachet adjacent the liquid, cream or gel-type product and opposite the closed end of the tubular sachet; and
sealing the remaining open end of the tubular sachet adjacent the insert.

10. Method of claim 9, wherein the step of sealing the applicator insert to the tubular sachet includes sealing the applicator insert along its periphery to the tubular foil such that no product can pass between the tubular foil and the applicator insert.

11. Method of claim 10, wherein the last step of sealing the opening at the second end and the step of sealing the applicator insert to the tubular sachet are performed at the same time.
